(19) United States
(12) Patent Application Publication
(10) Pub. No.: US 2021/0056563 A1
(43) Pub. Date: Feb. 25, 2021

(54) BIOMETRIC MEDICAL PROXIES

(71) Applicant: Alclear, LLC, New York, NY (US)

(72) Inventors: Marisa Bass, New York, NY (US); Joe Trelin, New York, NY (US)

(21) Appl. No.: 16/991,108

(22) Filed: Aug. 12, 2020

Related U.S. Application Data
(60) Provisional application No. 62/888,909, filed on Aug. 19, 2019.

(51) Int. Cl. G06Q 30/00; H04L 29/06; G16H 10/60; G16H 40/20; G16H 80/00; G16H 20/10; G06Q 30/06; G06K 9/00

(52) U.S. Cl. CPC G06Q 30/0185; H04L 63/0861; G16H 10/60; G16H 40/20; G06K 9/00892; G16H 20/10; G06Q 30/0623; G06Q 30/0633; G16H 80/00

(57) ABSTRACT

A system uses biometric identification to set and/or verify medical proxies. A proxy assignee may be granted a medical proxy for a proxy designator via the system by the proxy designator providing a digital representation of a biometric for the designator and/or specifying the rights to be granted and the identity of the assignee to whom to grant the rights. The assignee may accept the medical proxy by providing a digital representation of a biometric for the assignee, and the medical proxy may then be associated with identity information stored for the assignee. When the assignee requests to perform an action for the designator that requires an authorization, the system may identify the assignee. Upon determining that the medical proxy is associated with the identity (and/or verifying that the medical proxy authorizes the assignee for the action for the designator), the system may allow the action to be performed.

### CROSS-REFERENCE TO RELATED APPLICATION(S)

This application is a nonprovisional patent application of and claims the benefit of U.S. Patent Application No. 62/888,909, Aug. 19, 2019 and titled “Biometric Medical Proxies,” the disclosure of which is hereby incorporated herein by reference in its entirety.

### FIELD

The described embodiments relate generally to medical proxies. More particularly, the present embodiments relate to setting and verifying medical proxies using biometric identification systems.

### FIELD

The described embodiments relate generally to medical proxies. More particularly, the present embodiments relate to setting and verifying medical proxies using biometric identification systems.

### BACKGROUND

A person may create a proxy assignment that authorizes a person to perform an action on the person's behalf. Proxy assignments may authorize the other person to access information about the person, make decisions for the person, authorize actions to be performed on or for the person, and so on. These actions may be medical, financial, and so on. In some situations, proxy assignments may be made by operation of the law, such as where a law specifies that a parent or guardian has a proxy assignment to perform actions for a child or other ward.

For example, a person may be granted a medical proxy for another person. The medical proxy may authorize the person to make medical decisions for the other person, authorize medical procedures for the other person, refuse medical treatment for the other person, access medical records for the other person, communicate with medical service providers regarding medical care of the other person, and so on. The medical proxy may be general and authorize the person to make any medical decision for the other person, or may be limited and authorize the person to make specific medical decisions for the other person.

### SUMMARY

The present disclosure relates to setting and verifying medical proxies using biometric identification systems. A proxy assignee may be granted a medical proxy for a proxy designator by the proxy designator providing a digital representation of a biometric for the proxy designator to a biometric identification system and/or specifying the rights to be granted and the identity of the proxy assignee to whom to grant the rights. The proxy assignee may accept the medical proxy by providing a digital representation of a biometric for the proxy assignee, and the medical proxy may then be associated with identity information stored for the proxy assignee. When the proxy assignee requests to perform an action for the proxy designator that requires an authorization, the proxy assignee may be identified using the digital representation of the biometric for the proxy assignee. Upon determining that the medical proxy is associated with the identity (and/or verifying that the medical proxy authorizes the proxy assignee for the action for the proxy designator), the action may be performed.

In various embodiments, a biometric medical proxy system includes at least one non-transitory storage medium that stores instructions and at least one processing unit. The at least one processing unit executes the instructions to receive a request to perform an action that requires an authorization, determine that the authorization is obtained from a person asserting to have a medical proxy, determine an identity of the person using a digital representation of a biometric, and, upon determining that the medical proxy is associated with the identity, allow the action to be performed.

In some examples, the at least one processing unit determines the identity of the person by retrieving identity information that is associated with biometric data that matches the digital representation of the biometric. In various implementations of such examples, the identity information includes an assignment of the medical proxy to the person.

In a number of examples, the person is a proxy assignee and the action includes at least one of obtaining a medical product for a proxy designator, accessing a medical record of the proxy designator, communicating with a medical service provider regarding medical care provided to the proxy designator, or authorizing a medical procedure for the proxy designator. In various examples, the at least one processing unit verifies that the medical proxy authorizes the person to request the action prior to allowing the action to be performed.

In some examples, the at least one processing unit communicates with a first data store to determine the identity and a second data store to determine that the medical proxy is associated with the identity. In various implementations of such examples, the first data store is an identity system data store and the second data store is a medical records data store.

In some embodiments, a biometric medical proxy system includes at least one non-transitory storage medium that stores instructions and at least one processing unit. The at least one processing unit executes the instructions to receive a request to perform an action that requires an authorization from a particular person, obtain a digital representation of a biometric for the authorization, determine that an identity associated with the digital representation of the biometric is for a different person, and, upon determining that the identity is associated with a medical proxy regarding the action for the particular person (such that the different person may be a proxy assignee and the particular person may be a proxy designator), allow the action to be performed.

In various examples, the action includes picking up a prescription medical product. In some implementations of such examples, the at least one processing unit prompts the different person to select among prescription medical products available to pick up for multiple people for whom the different person has medical proxies.

In some examples, the at least one processing unit determines that the identity is associated with the medical proxy by transmitting a communication to the particular person to assign the medical proxy to the different person for the action. In a number of implementations of such examples, the digital representation of the biometric is a digital representation of a first biometric and the at least one processing unit receives a digital representation of a second biometric from the particular person to assign the medical proxy to the different person for the action. In various examples, the at least one processing unit performs the action.

In a number of embodiments, a biometric medical proxy system includes at least one non-transitory storage medium that stores instructions and at least one processing unit. The at least one processing unit executes the instructions to obtain a digital representation of a first biometric of a proxy designator and a request to designate a proxy assignee as a medical proxy for the proxy designator, determine a first identity of the proxy designator using the digital representation of the first biometric, obtain a digital representation of a second biometric from the proxy assignee, and assign the medical proxy to a second identity of the proxy assignee associated with the digital representation of the second biometric.

In some examples, the request to designate the proxy assignee as the medical proxy for the proxy designator may be the digital representation of the second biometric obtained by the at least one processing unit after the at least one processing unit obtains the digital representation of the first biometric. In various examples, the request to designate the proxy assignee as the medical proxy for the proxy designator specifies contact information for the proxy assignee and the at least one processing unit uses the contact information to request the digital representation of the second biometric from the proxy assignee. In some implementations of such examples, the contact information specifies an electronic device from which the digital representation of the second biometric can be obtained and that is associated with the proxy assignee. In other implementations of such examples, the contact information is used to determine the second identity, the second identity includes a specification of an electronic device from which the digital representation of the second biometric can be obtained and that is associated with the proxy assignee, and the at least one processing unit uses the specification to request the digital representation of the second biometric from the proxy assignee.

In some examples, the request to designate the proxy assignee as the medical proxy for the proxy designator specifies an action to which the medical proxy applies. In various examples, the at least one processing unit receives a request to perform an action that requires an authorization, receives the authorization from the proxy assignee, and, upon determining that the authorization includes the digital representation of the second biometric, allows the action to be performed.

### BRIEF DESCRIPTION OF THE DRAWINGS

The disclosure will be readily understood by the following detailed description in conjunction with the accompanying drawings, wherein like reference numerals designate like structural elements.

FIG. 1 depicts a first example of a biometric medical proxy system.

FIG. 2 is a flow chart illustrating a first example method for setting and/or verifying medical proxies using a biometric identification system. This method may be performed by the one or more of the systems of FIGS. 1 and/or 5-9.

FIG. 3 is a flow chart illustrating a second example method for setting and/or verifying medical proxies using a biometric identification system. This method may be performed by the one or more of the systems of FIGS. 1 and/or 5-9.

FIG. 4 is a flow chart illustrating a third example method for setting and/or verifying medical proxies using a biometric identification system. This method may be performed by the one or more of the systems of FIGS. 1 and/or 5-9.

FIG. 5 depicts a second example of a biometric medical proxy system.

FIG. 6 depicts a third example of a biometric medical proxy system.

FIG. 7 depicts a fourth example of a biometric medical proxy system.

FIG. 8A depicts a fifth example of a biometric medical proxy system.

FIG. 8B depicts the system of FIG. 8A when the proxy assignee accepts the medical proxy assignment.

FIG. 9 depicts a sixth example of a biometric medical proxy system.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, methods, and computer program products that embody various elements of the present disclosure. However, it should be understood that the described disclosure may be practiced in a variety of forms in addition to those described herein.

Medical proxies may be used for a variety of different reasons. For example, a person may be granted a medical proxy for another person so the person may communicate with medical service providers regarding care of the other person, access medical records of the other person, authorize surgeries and/or other medical procedures for the person, refuse medical treatment for the person, pick up prescription medication and/or other medical products for the person, and/or perform various other actions. The person may be granted authority to perform these actions in case the other person is unable to perform such actions himself (such as when the person is under anesthesia and/or otherwise incapacitated and/or otherwise unable to perform actions), in order to manage care for the other person, and so on.

Typically, a medical proxy may be a form that is completed by the other person and stored for later reference when used to verify authorization. For example, a medical service provider may obtain and store the medical proxy to verify that the person is authorized to perform an action for the other person. By way of another example, the person may store the medical proxy and provide it to verify authorization.

However, verifying the person's authorization to perform the action for the other person using a previously completed form may involve trusting that the other person specified by the form was actually the person who completed the form and that the person specified by the form is the correct person that the other person intended to grant authorization. For example, a medical service provider may reference a previously completed form when attempting to verify authorization. The medical service provider may be able to request a driver's license or other identification token at the time of verifying a medical proxy and verify a match to a name on a previously completed form in order to verify, but may be unable to ensure that the driver's license or other identification card is valid and/or that identity was sufficiently verified when the form was initially completed (e.g., that the other person granting the medical proxy correctly identified the person to whom the medical proxy was being granted, that the person to whom the medical proxy was being granted presented a valid driver's license or other identification card, and so on). Further, the medical service provider may lack the capability to verify the validity of the driver's license or other identification token. As such, the medical service provider may trust the previous identity verification and the driver's license or other identification token if both appear valid, even if they are not.

Additionally, a medical proxy recorded in a form or file in one medical service provider system may not be readily accessible and/or accessible by other medical service providers or systems. A doctor who stores such a form in a physical file at an office may have to spend the time to retrieve the form in order to verify the medical proxy, and may be unable to access the form from another location. Similarly, other medical service providers may be unable to access the form, severely limiting the use of the medical proxy and/or requiring a separate medical proxy to be completed for each medical service provider and/or system. In examples involving electronic medical service provider systems, this may involve additional data storage equipment to store duplicate data, increased processor and/or network traffic burden to support storage and access for storing and locating separate medical proxies, and so on.

The following disclosure relates to setting and verifying medical proxies using biometric identification systems. A proxy assignee may be granted a medical proxy for a proxy designator by the proxy designator providing a digital representation of a biometric for the proxy designator to a biometric identification system and/or specifying the rights to be granted and the identity of the proxy assignee to whom to grant the rights. The proxy assignee may accept the medical proxy by providing a digital representation of a biometric for the proxy assignee, and the medical proxy may then be associated with identity information stored for the proxy assignee. When the proxy assignee requests to perform an action for the proxy designator that requires an authorization, the proxy assignee may be identified using the digital representation of the biometric for the proxy assignee. Upon determining that the medical proxy is associated with the identity (and/or verifying that the medical proxy authorizes the proxy assignee for the action for the proxy designator), the action may be performed.

In this way, any medical service provider and/or system that uses the biometric identification system to identify the person and/or the other person may be able to access the medical proxy because it is associated with the identity information stored for the person. This may allow the systems to perform functions that they were not previously able to perform, as well as improving the efficiency and operation of the systems by reducing duplicate data storage equipment, decreasing processor and/or network traffic burden to support storage, and so on. This may also improve security and accuracy as the identities of the people involved may be biometrically ascertained and/or verified.

In various examples, a biometric medical proxy system may receive a request to perform an action that requires an authorization. The biometric medical proxy system may determine that the authorization is obtained from a person asserting to have a medical proxy and determine an identity of the person using a digital representation of a biometric. Upon determining that the medical proxy is associated with the identity, the biometric medical proxy system may allow the action to be performed.

In some examples, a biometric medical proxy system may receive a request to perform an action that requires an authorization from a particular person. The biometric medical proxy system may obtain a digital representation of a biometric for the authorization and determine that an identity associated with the digital representation of the biometric is for a different person. Upon determining that the identity is associated with a medical proxy regarding the action for the particular person, the biometric medical proxy system may allow the action to be performed.

In a number of examples, a biometric medical proxy system may obtain a digital representation of a first biometric of a proxy designator and a request to designate a proxy assignee as a medical proxy for the proxy designator. The biometric medical proxy system may determine a first identity of the proxy designator using the digital representation of the first biometric, obtain a digital representation of a second biometric from the proxy assignee, and assign the medical proxy to a second identity of the proxy assignee associated with the digital representation of the second biometric.

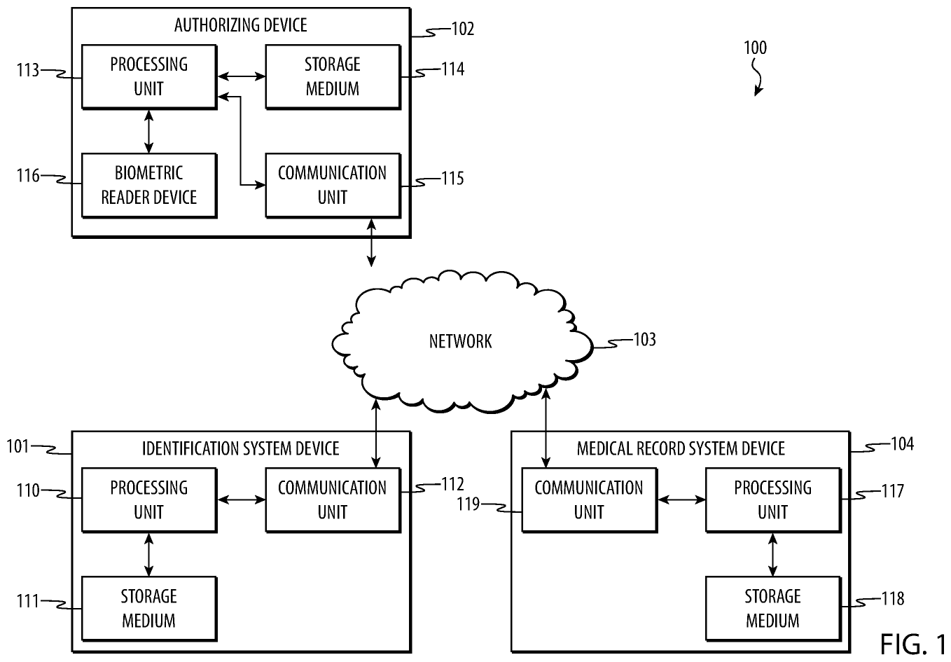

These and other embodiments are discussed below with reference to FIGS. 1-9. However, those skilled in the art will readily appreciate that the detailed description given herein with respect to these Figures is for explanatory purposes only and should not be construed as limiting.

FIG. 1 depicts a first example of a biometric medical proxy system 100. The system 100 may include one or more identification system devices 101 that are operable to communicate with one or more authorizing devices 102 and/or other devices (such as a medical records system device 104) via one or more networks 103. The system 100 may use one or more of these components to perform various actions related to setting and verifying medical proxies using one or more biometric identification systems.

A person may be granted a medical proxy for another person by the other person providing a digital representation of a biometric for the other person to the identification system device 101 via the authorizing device 102 and/or one or more other electronic devices (such as one or more personal electronic devices, enrollment devices, and so on) and/or specifying the rights to be granted and the identity of the person to whom to grant the rights. The person may accept the medical proxy by providing a digital representation of a biometric for the person to the identification system device 101 (which may store identity information and biometric data and/or other identification information for people and identify people by matching received digital representations of biometrics to biometric data associated with specific identity information) via the authorizing device 102 and/or the one or more other electronic devices, and the medical proxy may then be associated with identity information stored for the person. For example, the medical proxy may be stored in identity information for the person, in identity information for the other person and accessible through a link in the identity information for the person, in one or more medical record repositories hosted by the medical records system device 104 and accessible through a link in the identity information for the person, and so on.

When the authorizing device 102 receives a request from the person to perform an action for the other person that requires an authorization, the authorizing device 102 may identify the person by receiving the digital representation of the biometric of the person and submitting the digital representation of the biometric of the person to the identification system device 101. The identification system device 101 may determine the identity of the person using the digital representation of the biometric for the person and provide and/or otherwise access medical proxy information associated with identity information stored for the person. Upon the authorizing device 102 determining that the medical proxy is associated with the identity of the person, the authorizing device 102 may perform and/or otherwise allow the action to be performed.

In this way, any device that uses the system 100 to identify the person and/or the other person may be able to access the medical proxy because it is associated with the identity information stored for the person. This may enable the system 100 to perform functions that the system 100 would not otherwise be able to perform, as well as improving the efficiency and operation of the system 100 by reducing duplicate data storage equipment, decreasing processor and/or network traffic burden to support storage, and so on. This may also improve security and accuracy as the identities of the people involved may be biometrically ascertained and/or verified.

For example, a doctor may provide a tablet computing device to a person who wishes to act as a proxy designator and grant a medical proxy to a proxy assignee. The proxy designator may identify himself by using the tablet computing device to take a picture of his face and specifying the rights to be granted (and/or specifying all rights and/or a default set of rights by not directly specifying the rights to be granted). The proxy designator may specify a proxy assignee to assign the medical proxy to by handing the tablet computing device to the proxy assignee, who may identify herself by using the tablet computing device to take a picture of her face. The medical proxy may then be stored in association with identity information for the proxy assignee.

For example, the medical proxy may be stored in a data structure associated with the identity information that includes one or more data fields specifying one or more actions authorized to be performed (whether or not under various conditions) on behalf of one or more proxy designators, one or more links to corresponding data stored in identity information associated with one or more corresponding proxy designators, and so on. In various examples, a similar data structure may be additionally and/or alternatively stored in identity information associated with the respective proxy designator. Various configurations are possible and contemplated without departing from the scope of the present disclosure.

Subsequently, the proxy assignee may request an action from the doctor for the proxy designator (such as to communicate with the doctor regarding care of the proxy designator, to access medical records of the proxy designator, to make a decision regarding care of the proxy designator, to refuse care for the proxy designator, to pick up prescription medication and/or other medical products for the proxy designator, and so on). The proxy assignee may identify herself by allowing the tablet computing device and/or another electronic device to capture an image of her face. Upon identification, information regarding the medical proxy associated with the identity information of the proxy assignee may be retrieved and the action may be performed and/or allowed to be performed. Various configurations are possible and contemplated without departing from the scope of the present disclosure.

By way of another example, a proxy designator may use a first personal computing device to identify himself by using the first personal computing device to take a picture of his face, specifying the rights to be granted in a medical proxy, and specifying a proxy assignee to whom to assign the medical proxy. Contact information for the proxy assignee may be accessed from identity information stored for the proxy assignee (such as an email address, a phone number, an identifier of an electronic device associated with the proxy assignee, and so on) and used to contact the proxy assignee to accept assignment of the medical proxy. The proxy assignee may use a second personal computing device to identify herself by using the second personal computing device to take a picture of his face. The medical proxy may then be stored in association with identity information for the proxy assignee.

Subsequently, the proxy assignee may request an action for the proxy designator (such as to communicate with a medical service provider regarding care of the proxy designator, to access medical records of the proxy designator, to make a decision regarding care of the proxy designator, to refuse care for the proxy designator, to pick up prescription medication and/or other medical products for the proxy designator, and so on). The proxy assignee may identify herself by allowing an electronic device to capture an image of her face. Upon identification, information regarding the medical proxy associated with the identity information of the proxy assignee may be retrieved and the action may be performed and/or allowed to be performed. Various configurations are possible and contemplated without departing from the scope of the present disclosure.

In yet another example, a first person who happens to be a proxy assignee for a second person (i.e., a proxy designator) may submit a request at an automated pharmacy to pick up a prescription and/or other medical product. The proxy assignee may provide a fingerprint to a biometric reader device. The identity of the proxy assignee may be determined using a digital representation of the fingerprint and it may be determined that the prescription is for the proxy designator instead of the proxy assignee. Identity information for the proxy assignee may be accessed, which may indicate that the proxy assignee has a medical proxy for the proxy designator. As such, the proxy assignee may be allowed to pick up the prescription. Various configurations are possible and contemplated without departing from the scope of the present disclosure.

In still another example, a proxy assignee may submit a request at an automated pharmacy to pick up a prescription and/or other medical product for a proxy designator. The proxy assignee may provide a fingerprint to a biometric reader device. The identity of the proxy assignee may be determined using a digital representation of the fingerprint and identity information for the proxy assignee may be accessed, which may indicate that the proxy assignee has a medical proxy for the proxy designator. As such, the proxy assignee may be allowed to pick up the prescription. Various configurations are possible and contemplated without departing from the scope of the present disclosure.

In a further example, a person may provide a fingerprint to a biometric reader device at an automated pharmacy. The identity of the person may be determined using a digital representation of the fingerprint and it may be determined that the person has one or more medical proxies for one or more other people indicated in the person's identity information. One or more prescriptions and/or other medical products may be identified as available for pickup by the person and/or the one or more other people for whom the person has a medical proxy. The person may then be prompted to select one or more of the prescriptions to pick up and the selected prescriptions may be provided to the person. In some implementations, identity information stored for the person and/or the other people may be used to charge one or more financial accounts, insurance, and/or other payment sources associated with the person and/or the other people as specified in the identity information stored for the person and/or the other people. Various configurations are possible and contemplated without departing from the scope of the present disclosure.

In still a further example, a person may submit a request at an automated pharmacy to pick up a prescription and/or other medical product for another person. The person may provide a fingerprint to a biometric reader device. The identity of the person may be determined using a digital representation of the fingerprint and identity information for the person may be accessed, which may not indicate that the person has a medical proxy for the other person. Contact information for the other person may then be retrieved that is associated with identity information for the other person and used to transmit a message to an electronic device associated with the other person to request a medical proxy for the person that covers the prescription. The other person may identify herself by capturing an image of her face using an imaging device of the electronic device. This may grant the medical proxy, which may then be associated with the identity information of the person (i.e., the proxy assignee) and/or the identity information of the other person (i.e., the proxy designator). The proxy assignee may be then allowed to pick up the prescription. Various configurations are possible and contemplated without departing from the scope of the present disclosure.

In various implementations, the system 100 may receive a request to perform an action that requires an authorization. The system 100 may determine that the authorization is obtained from a person asserting to have a medical proxy and determine an identity of the person using a digital representation of a biometric. Upon determining that the medical proxy is associated with the identity, the system 100 may allow the action to be performed.

In some implementations, the system 100 may receive a request to perform an action that requires an authorization from a particular person. The system 100 may obtain a digital representation of a biometric for the authorization and determine that an identity associated with the digital representation of the biometric is for a different person. Upon determining that the identity is associated with a medical proxy regarding the action for the particular person, the system 100 may allow the action to be performed.

In a number of implementations, the system 100 may obtain a digital representation of a first biometric of a proxy designator and a request to designate a proxy assignee as a medical proxy for the proxy designator. The system 100 may determine a first identity of the proxy designator using the digital representation of the first biometric, obtain a digital representation of a second biometric from the proxy assignee, and assign the medical proxy to a second identity of the proxy assignee associated with the digital representation of the second biometric.

In some examples, medical proxies may be granted with a limited time of use. For example, the medical proxy may be granted with a specific expiration date, a specific number of uses (such as a medical proxy that can be used once and/or another specific number of times), and so on. When the designated time and/or number of use for the medical proxy is reached, the system 100 may disable and/or otherwise revoke the medical proxy. Various configurations are contemplated and possible without departing from the scope of the present disclosure.

In some such examples, medical proxies granted with a limited use may be renewable. For example, a proxy designator may request renewal when the medical proxy may be ready to expire and/or has expired. By way of another example, the system 100 may detect possible and/or actual expiration, contact the proxy designator to indicate possible and/or actual expiration, and prompt for a renewal request. Regardless, upon receipt of an indication to renew, the system 100 may contact the proxy assignee to inform the proxy assignee about renewal, prompt the proxy assignee to accept the renewal, and/or perform various other actions. Various configurations are contemplated and possible without departing from the scope of the present disclosure.

Although the above discusses assignment and use of medical proxies, it is understood that these are examples. In various implementations, assigned medical proxies may be revocable. For example, in some implementations, the system 100 may receive a request from a proxy designator to revoke a medical proxy that was assigned to a proxy assignee. The system 100 may determine the identity of the proxy designator, determine the identity of the proxy assignee, and revoke the assignment of the medical proxy. By way of illustration, a medical proxy stored in identity data for the proxy assignee may be linked to identity data stored for the proxy designator (which may include links to all medical proxies granted by the proxy designator to various proxy assignees). Upon revocation by the proxy designator, the link may be followed to revoke the medical proxy stored in the identity data for the proxy assignee. Various configurations are contemplated and possible without departing from the scope of the present disclosure.

The identification system device 101 may be any kind of electronic device and/or cloud and/or other computing arrangement. Examples of such devices include, but are not limited to, one or more desktop computing devices, laptop computing devices, server computing devices, mobile computing devices, wearable devices, tablet computing devices, mobile telephones, smart phones, printers, displays, kiosks, vehicles, kitchen appliances, entertainment system devices, digital media players, and so on. The identification system device 101 may include one more processing units 110 and/or other processors or controllers, non-transitory storage media 111 (which may take the form of, but is not limited to, a magnetic storage medium; optical storage medium; magneto-optical storage medium; read only memory; random access memory; erasable programmable memory; flash memory; and so on), communication units 112, and/or other components. The processing unit 110 may execute one or more sets of instructions stored in the non-transitory storage medium 111 to perform various functions, such as storing biometric data and/or other identification information for people and associated identity information (such as one or more names, addresses, telephone numbers, financial data, financial account numbers, verified ages, insurance identifiers, payment account identifiers, medical proxies, and so on) in a data store (such as an identity system data store), receiving one or more digital representations of biometrics, matching one or more received digital representations of biometrics to stored biometric data, retrieving identity information associated with stored biometric data matching one or more received digital representations of biometrics, providing retrieved identity information, communicating with the authorizing device 102 and/or the medical records system device 104 and/or one or more other electronic devices via the network 103 using the communication unit 112, and so on.

In some examples, the identification system device 101 may store identity information associated with identification information (such as biometric data, logins, passwords, account identifiers, and so on) and may use the identification information to control access to the identity information. For example, the identification system device 101 may be operative to receive one or more digital representations of biometrics (such as one or more hashes and/or other digital representations of one or more fingerprints, blood vessel scans, palm-vein scans, voiceprints, facial images, retina images, iris images, deoxyribonucleic acid sequences, heart rhythms, gaits, and so on) for one or more people, determine one or more identities by comparing the digital representations of biometrics to stored biometric data associated with the identity information, and provide information about the determined identities (such as identifying the identity, providing information stored in the identity information, providing attestations about the identity, and so on).

Likewise, the authorizing device 102 may be any kind of electronic device and may include one or more processing units 113, non-transitory storage medium 114, communication units 115, biometric reader devices 116 (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on), and so on. The processing unit 113 may execute one or more sets of instructions stored in the non-transitory storage medium 114 to perform various functions, such as obtaining one or more digital representations of biometrics via the biometric reader device 116, receiving requests for actions (that may or may not require authorization), determining whether or not a person is authorized for a requested action (such as whether or not the person is the person for whom the action is to be performed and is authorized to request the action for himself, whether or not the person has a medical proxy authorizing the action, and so on), communicating with the identification system device 101 and/or the medical records system device 104 and/or one or more other electronic devices via the network 103 using the communication unit 115, and so on. The authorizing device 102 may “authorize” by authorizing actions based on medical proxies, authorizing assignment of medical proxies, and so on.

Similarly, the medical records system device 104 may be any kind of electronic device and may include one or more processing units 117, non-transitory storage medium 118, communication units 119, and so on. The processing unit 117 may execute one or more sets of instructions stored in the non-transitory storage medium 118 to perform various functions, such as storing one or more medical records in a data store (such as a medical records data store), providing one or more medical records (such as to the identification system device 101 in response to a request from the identification system device 101, to the authorizing device 102 in response to a request from the identification system device 101, and so on), communicating with the identification system device 101 and/or the authorizing device 102 and/or one or more other electronic devices via the network 103 using the communication unit 119, and so on.

Although the system 100 is illustrated and described as including particular components arranged in a particular configuration, it is understood that this is an example. In other implementations, other configurations of the same, similar, and/or different components may be used without departing from the scope of the present disclosure.

For example, the system 100 is illustrated and described as including the authorizing device 102. However, in other examples, a medical proxy assignment device and/or an enrollment device and/or other device may be included in addition to and/or instead of the authorizing device 102. Various implementations are possible and contemplated without departing from the scope of the present disclosure.

Further, although the system 100 is illustrated and described in the context of a medical proxy, it is understood that this is an example. In various implementation, proxies granting rights for any kind of action (such as financial) may be set and/or verified without departing from the scope of the present disclosure.

For example, a proxy designator may assign various use permissions to a proxy assignee with respect to a credit card and/or other financial account. This may enable the proxy assignee to use the credit card, whether or not under one or more different conditions. By way of illustration, a parent may grant a child a proxy to use a credit card to go to a grocery store but not a liquor store, to use the credit card for 24 hours, and so on. Various configurations are possible and contemplated without departing from the scope of the present disclosure.

By way of another example, proxies may be used to clarify confusion between real and apparent authority for a corporation or other entity. A company corporate board may assign one or more proxies to one or more corporate officers granting those corporate officers (such as a chief operating officer, a chief financial officer, a vice president of engineering, and so on) authority to perform one or more actions on behalf of the company. These corporate officers may then in turn re-assign part or all of the proxy authority to sub-officers (such as the chief operating officer delegating authority to perform one or more actions authorized by the company corporate board to the chief financial officer, the vice president of engineering, and so on). This may provide verifiable evidence regarding who is actually authorized to perform various actions for the company, as well as possibly being used to create an audit trail for verifying who performed what actions and/or whether or not that person was authorized and/or who provided that authority. Various configurations are possible and contemplated without departing from the scope of the present disclosure.

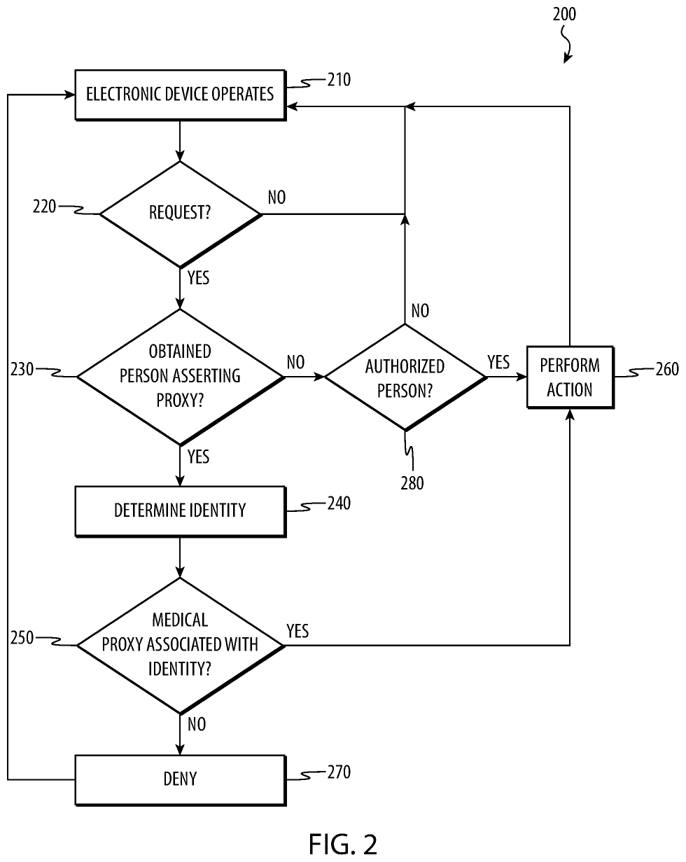

FIG. 2 is a flow chart illustrating a first example method 200 for setting and/or verifying medical proxies using a biometric identification system. This method 200 may be performed by the one or more of the systems 100, 500, 600, 700, 800, 900 of FIGS. 1 and/or 5-9.

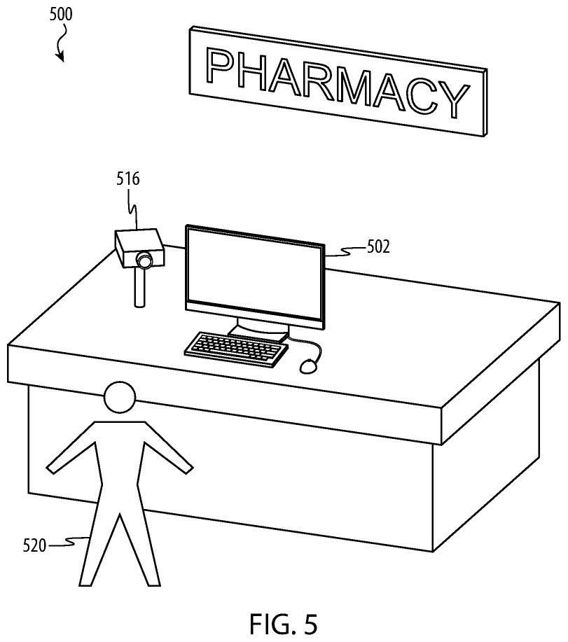

At operation 210, an electronic device (such as the identification system device 101 of FIG. 1; the medical record system device 104 of FIG. 1; the authorizing device 102 of FIG. 1, the electronic devices 502, 602, 702, 802, 902A, and 902B of FIGS. 5-9 discussed below; and so on) may operate. The flow may proceed to operation 220 where the electronic device may determine whether or not a request to perform an action that requires authorization may be received. Such an action may be an action to make a medical decision for a person, authorize a medical procedure for a person, refuse medical treatment for a person, access a medical record for a person, communicate with a medical service provider regarding medical care of a person, pick up a prescription or other medical product for a person, and so on. If so, the flow may proceed to operation 230. Otherwise, the flow may return to operation 210 where the electronic device may continue to operate.

At operation 230, after the electronic device determines that the request is received, the electronic device may determine whether or not the request is obtained from a person asserting a proxy, such as a medical proxy. For example, the electronic device may determine that the person is asserting a proxy if the person specifies that he is submitting the request on behalf of another person. By way of another example, the electronic device may determine that the person is asserting a proxy if the person is submitting a request that requires authorization by another person. If so, the flow may proceed to operation 240. Otherwise, the operation may proceed to operation 280.

At operation 240, after the electronic device determines that the request is obtained from a person asserting a proxy, the electronic device may determine an identity of the person. For example, the electronic device may obtain a digital representation of a biometric for a person and/or other information and submit such to an identification system that matches the digital representation of the biometric to stored biometric data and/or other identification information associated with identity information stored for the identity of the person.

The flow may then proceed to 250 where the electronic device may determine whether or not a medical proxy covering the request is associated with the identity. For example, the identity information stored for the identity may include an assignment of the medical proxy. By way of another example, the identity information stored for the identity may include a link to an assignment of the medical proxy stored in association with the identity of another person. By way of still another example, the identity information stored for the identity may include a link to an assignment of the medical proxy stored in a medical record repository.

If so, the flow may proceed to operation 260 where the electronic device may perform the action and/or allow the action to be performed before the flow returns to operation 210 and the electronic device continues to operate. Otherwise, the flow may proceed to operation 270 where the electronic device may deny the action and/or prevent the action from being performed before the flow returns to operation 210 and the electronic device continues to operate.

At operation 280, after the electronic device determines that the request is not obtained from a person asserting a proxy, the electronic device may determine whether or not the person is authorized to request the action. If so, the flow may proceed to operation 260 where the electronic device may perform the action and/or allow the action to be performed before the flow returns to operation 210 and the electronic device continues to operate. Otherwise, the flow may return directly to operation 210 where the electronic device continues to operate.

In various examples, this example method 200 may be implemented as a group of interrelated software modules or components that perform various functions discussed herein. These software modules or components may be executed within a cloud network and/or by one or more computing devices, such as the identification system device 101 of FIG. 1; the medical record system device 104 of FIG. 1; the authorizing device 102 of FIG. 1, the electronic devices 502, 602, 702, 802, 902A, and 902B of FIGS. 5-9 discussed below; and so on.

Although the example method 200 is illustrated and described as including particular operations performed in a particular order, it is understood that this is an example. In various implementations, various orders of the same, similar, and/or different operations may be performed without departing from the scope of the present disclosure.

For example, the method 200 is illustrated and described as determining whether or not the person submitting the request is asserting a proxy. However, in other examples, the electronic device may determine whether or not the person is authorized to submit the request without consideration of any proxy assertion. In some such examples, the person may be authorized if the person is either the person for whom the action is requested or the person has a proxy for the person on whose behalf the action is requested.

By way of another example, the method 200 is illustrated and described as receiving requests for actions that require authorization. However, in other examples, some requests for actions may require authorizations whereas other actions do not require authorizations. In such an example, the electronic device may perform and/or allow performance of actions not requiring authorizations without determining whether or not the person is authorized and/or has a proxy. Various configurations are possible and contemplated without departing from the scope of the present disclosure.

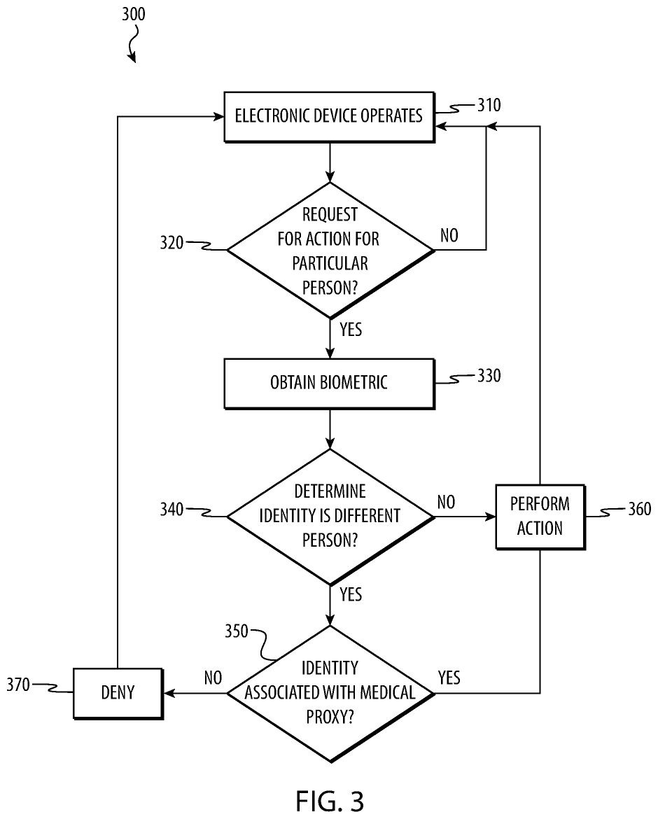

FIG. 3 is a flow chart illustrating a second example method 300 for setting and/or verifying medical proxies using a biometric identification system. This method 300 may be performed by the one or more of the systems 100, 500, 600, 700, 800, 900 of FIGS. 1 and/or 5-9.

At operation 310, an electronic device (such as the identification system device 101 of FIG. 1; the medical record system device 104 of FIG. 1; the authorizing device 102 of FIG. 1, the electronic devices 502, 602, 702, 802, 902A, and 902B of FIGS. 5-9 discussed below; and so on) may operate. The flow may proceed to operation 320 where the electronic device may determine whether or not a request to perform an action for a particular person is received. If so, the flow may proceed to operation 330. Otherwise, the flow may return to operation 310 where the electronic device may continue to operate.

At operation 330, after the electronic device determines that the request to perform an action for a particular person is obtained, the electronic device may obtain a digital representation of a biometric from the person who submitted the request. The flow may then proceed to operation 340 where the electronic device may use the digital representation of the biometric to determine whether or not the identity of the person who submitted the request is the identity of a different person rather than the particular person. If not, the flow may proceed to operation 360 where the electronic device may perform the action and/or allow the action to be performed before the flow returns to operation 310 and the electronic device continues to operate. Otherwise, the flow may proceed to operation 350.

At operation 350, after the electronic device determines that the identity of the person who submitted the request is the identity of a different person rather than the particular person, the electronic device may determine whether or not the identity is associated with a medical proxy for the particular person (i.e., the particular person is a proxy designator and the different person is a proxy assignee of the proxy designator). If so, the flow may proceed to operation 360 where the electronic device may perform the action and/or allow the action to be performed before the flow returns to operation 310 and the electronic device continues to operate. Otherwise, the flow may proceed to operation 370 where the electronic device may deny the action and/or prevent the action from being performed before the flow returns to operation 310 and the electronic device continues to operate.

In various examples, this example method 300 may be implemented as a group of interrelated software modules or components that perform various functions discussed herein. These software modules or components may be executed within a cloud network and/or by one or more computing devices, such as the identification system device 101 of FIG. 1; the medical record system device 104 of FIG. 1; the authorizing device 102 of FIG. 1, the electronic devices 502, 602, 702, 802, 902A, and 902B of FIGS. 5-9 discussed below; and so on.

Although the example method 300 is illustrated and described as including particular operations performed in a particular order, it is understood that this is an example. In various implementations, various orders of the same, similar, and/or different operations may be performed without departing from the scope of the present disclosure.

For example, the method 300 is illustrated and described as determining whether or not the request is received to perform an action for a particular person. However, in other examples, the request may not specify a person for whom the action is to be performed. In some examples, the request may specify the action and the electronic device may determine a particular person who has authority to perform the action. The electronic device may then determine whether or not the request is received from that particular person or from a different person who has an appropriate proxy in order to perform and/or allow performance of the action. Various configurations are possible and contemplated without departing from the scope of the present disclosure.

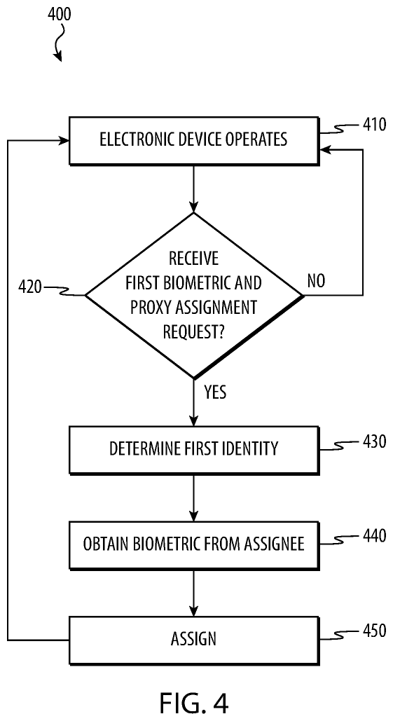

FIG. 4 is a flow chart illustrating a third example method 400 for setting and/or verifying medical proxies using a biometric identification system. This method 400 may be performed by the one or more of the systems 100, 500, 600, 700, 800, 900 of FIGS. 1 and/or 5-9.

At operation 410, an electronic device (such as the identification system device 101 of FIG. 1; the medical record system device 104 of FIG. 1; the authorizing device 102 of FIG. 1, the electronic devices 502, 602, 702, 802, 902A, and 902B of FIGS. 5-9 discussed below; and so on) may operate. The flow may proceed to operation 420 where the electronic device may determine whether or not a digital representation of a first biometric and a proxy assignment are received. If so, the flow may proceed to operation 430. Otherwise, the flow may return to operation 410 where the electronic device may continue to operate.

At operation 430, the electronic device may use the digital representation of the biometric to determine a first identity. This first identity may be the identity over which the proxy assignment is to assign rights. The rights may be all that the first identity is able to grant and/or a subset thereof.

At operation 440, the electronic device may obtain a second digital representation of a second biometric from the assignee of the proxy assignment request. This may confirm the assignee's identity and/or confirm that the assignee accepts the proxy assignment. For example, the proxy assignment request may specify identification information for the assignee that is then used to contact the assignee in order to obtain the digital representation of the second biometric from the assignee. By way of another example, the person providing the assignment request may provide the digital representation of the first biometric using an electronic device and then provide the electronic device to the assignee to use to provide the digital representation of the second biometric, signaling that the assignee is the person to whom the person has provided the electronic device. Various configurations are possible and contemplated without departing from the scope of the present disclosure.

At operation 450, the electronic device may assign the proxy to the assignee. The proxy may be stored in identity information for the assignee, in identity information for the person and accessible through a link in the identity information for the assignee, in one or more record repositories accessible through a link in the identity information for the assignee, and so on.

In various examples, this example method 400 may be implemented as a group of interrelated software modules or components that perform various functions discussed herein. These software modules or components may be executed within a cloud network and/or by one or more computing devices, such as the identification system device 101 of FIG. 1; the medical record system device 104 of FIG. 1; the authorizing device 102 of FIG. 1, the electronic devices 502, 602, 702, 802, 902A, and 902B of FIGS. 5-9 discussed below; and so on.

Although the example method 400 is illustrated and described as including particular operations performed in a particular order, it is understood that this is an example. In various implementations, various orders of the same, similar, and/or different operations may be performed without departing from the scope of the present disclosure.

For example, the method 400 is illustrated and described as obtaining the digital representation of the second biometric from the assignee and assigning the proxy. However, in some examples, the assignee may not be identifiable from the proxy assignment request and/or the assignee may refuse the assignment. In such examples, the electronic device may determine that an error condition has occurred and prompt the person who submitted the request accordingly. Various configurations are possible and contemplated without departing from the scope of the present disclosure.

FIG. 5 depicts a second example of a biometric medical proxy system 500. The system 500 may include an electronic device 502 at an automated pharmacy that a person 520 may use to submit a request to pick up a prescription and/or other medical product on behalf of another person. As part of submitting the request, the person 520 may provide a digital representation of a biometric via a biometric reader device 516 associated with the electronic device. For example, the biometric reader device 516 may be a camera or other imaging device that captures an image of the face of the person 520 and/or a portion thereof.

In one example, the identity of the person 520 may be determined using the digital representation of the biometric and it may be determined that the prescription is for another person. Identity information for the person 520 may be accessed, which may indicate that the person 520 has a medical proxy for the other person. As such, the person 520 may be allowed to pick up the prescription (such as by triggering one or more automated dispensing mechanisms, signaling one or more pharmacy personnel, and so on). Various configurations are possible and contemplated without departing from the scope of the present disclosure.

In another example, the identity of the person 520 may be determined using the digital representation of the biometric and identity information for the person 520 may be accessed, which may indicate that the person 520 has a medical proxy for another person. As such, the person 520 may be allowed to pick up a prescription for the other person. Various configurations are possible and contemplated without departing from the scope of the present disclosure.

In a third example, the identity of the person 520 may be determined using the digital representation of the biometric and it may be determined that the person 520 has one or more medical proxies for one or more other people indicated in the person's identity information. One or more prescriptions and/or other medical products may be identified as available for pickup by the person 520 and/or the one or more other people for whom the person 520 has a medical proxy. The person 520 may then be prompted to select one or more of the prescriptions to pick up and the selected prescriptions may be provided to the person 520. In some implementations, identity information stored for the person 520 and/or the other people may be used to charge one or more financial accounts, insurance, and/or other payment sources associated with the person 520 and/or the other people as specified in the identity information stored for the person and/or the other people. Various configurations are possible and contemplated without departing from the scope of the present disclosure.

In a fourth example, the identity of the person 520 may be determined using the digital representation of the biometric and identity information for the person 520 may be accessed, which may not indicate that the person 520 has a medical proxy for another person associated with a requested prescription and/or other medical product. Contact information for the other person may then be retrieved that is associated with identity information for the other person and used to transmit a message to an electronic device associated with the other person to request a medical proxy for the person 520 that covers the prescription. The other person may identify herself by capturing an image of her face using an imaging device of the electronic device. This may grant the medical proxy, which may then be associated with the identity information of the other person and/or the identity information of the person 520. The person 520 may be then allowed to pick up the prescription. Various configurations are possible and contemplated without departing from the scope of the present disclosure.

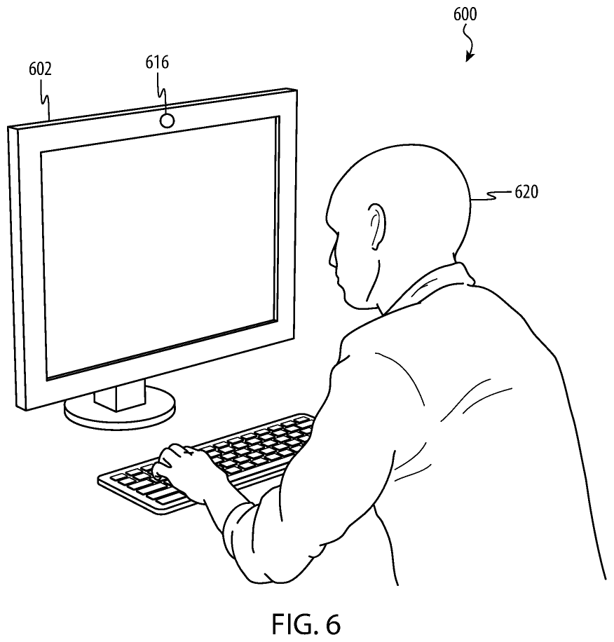

FIG. 6 depicts a third example of a biometric medical proxy system 600. The system 600 may include an electronic device 602 that a person 620 uses to submit a request to access a medical record of another person. As part of requesting to access the medical record, the person 620 may provide a digital representation of a biometric via a biometric reader device 616 associated with the electronic device 602. For example, the biometric reader device 616 may be a camera or other imaging device that captures an image of the face of the person 620 and/or a portion thereof. The identity of the person 620 may be determined using the digital representation of the biometric and identity information for the person 620 may be accessed, which may indicate that the person 620 has a medical proxy for the other person. As such, the person 620 may be allowed to access the medical records of the other person. Various configurations are possible and contemplated without departing from the scope of the present disclosure. Various configurations are possible and contemplated without departing from the scope of the present disclosure.

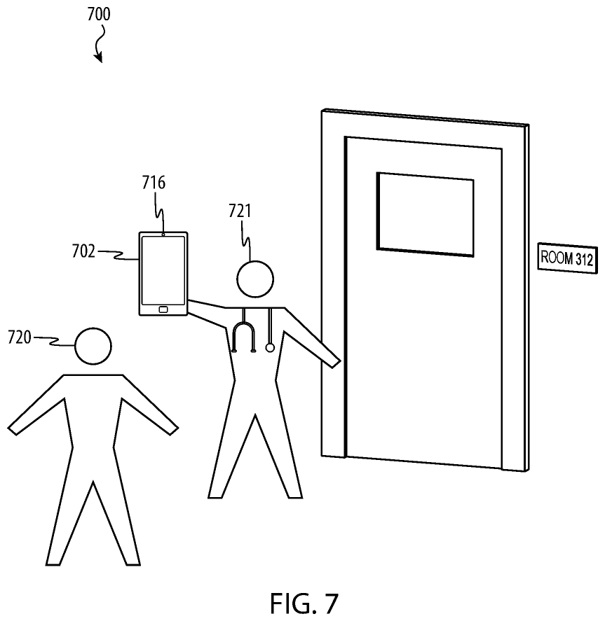

FIG. 7 depicts a fourth example of a biometric medical proxy system 700. The system 700 may include an electronic device 702 that a medical service provider 721 may use outside the hospital room of a person to evaluate a request from another person 720 to communicate regarding the person's medical care. As part of evaluating the request, the medical service provider 721 may use the electronic device 702 to obtain a digital representation of a biometric from the other person 720 via a biometric reader device 716 associated with the electronic device 702. For example, the biometric reader device 716 may be a camera or other imaging device that captures an image of the face of the other person 720 and/or a portion thereof. The identity of the other person 720 may be determined using the digital representation of the biometric and identity information for the other person 720 may be accessed, which may indicate that the other person 720 has a medical proxy for the person. As such, medical service provider 721 may communicate with the other person 720 regarding the person's medical care. Various configurations are possible and contemplated without departing from the scope of the present disclosure.

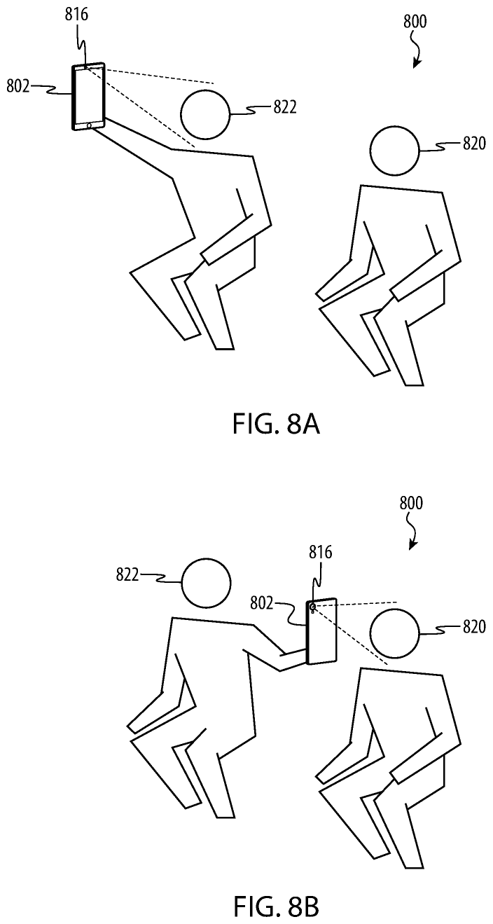

FIG. 8A depicts a fifth example of a biometric medical proxy system 800. The system 800 may include an electronic device 802 that a proxy designator 822 may use to identify himself by providing a digital representation of a first biometric via a biometric reader device 816 of the electronic device 802 and to provide a proxy request to assign a proxy. For example, the biometric reader device 816 may be a camera or other imaging device that captures an image of the face of the proxy designator 822 and/or a portion thereof. As shown in FIG. 8B, the proxy designator 822 may then use the electronic device 802 to capture a digital representation of a second biometric of a proxy assignee 820, indicating that the proxy assignee 820 is the assignee of the proxy (though in some implementations the proxy request may specify the proxy assignee 820 specifically). Various configurations are possible and contemplated without departing from the scope of the present disclosure.

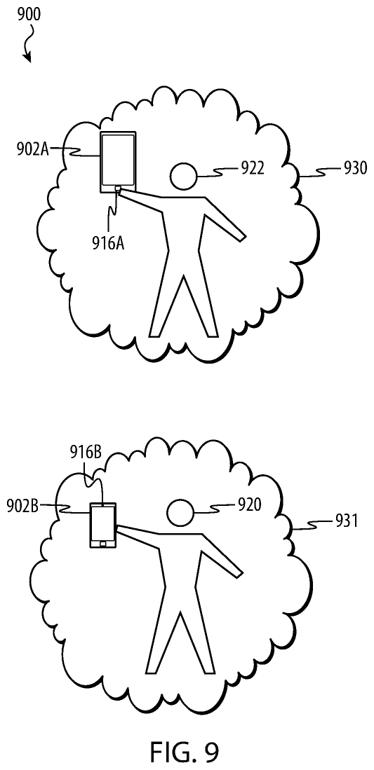

FIG. 9 depicts a sixth example of a biometric medical proxy system 900. The system 900 may include a first electronic device 902A that a proxy designator 922 may use at a first location 930 to identify himself by providing a digital representation of a first biometric via a biometric reader device 916A of the first electronic device 902A and to provide a proxy request to assign a proxy. For example, the biometric reader device 916A may be a fingerprint sensor that captures an image of a fingerprint of the proxy designator 922. The system 900 may also include a second electronic device 902B that a proxy assignee 920 may use at a second location 931 to identify himself by providing a digital representation of a second biometric via a biometric reader device 916B in order to accept the assignment. For example, the biometric reader device 916B may be a camera or other imaging device that captures an image of the face of the proxy assignee 920 and/or a portion thereof. In some examples, the proxy designator 922 may specify contact information or the proxy assignee 920 with the proxy request. In other examples, contact information for the proxy assignee 920 may be retrieved from identity information stored for the proxy assignee 920. Various configurations are possible and contemplated without departing from the scope of the present disclosure.

In various implementations, a biometric medical proxy system may include at least one non-transitory storage medium that stores instructions and at least one processing unit. The at least one processing unit may execute the instructions to receive a request to perform an action that requires an authorization, determine that the authorization is obtained from a person asserting to have a medical proxy, determine an identity of the person using a digital representation of a biometric, and, upon determining that the medical proxy is associated with the identity, allow the action to be performed.

In some examples, the at least one processing unit may determine the identity of the person by retrieving identity information that is associated with biometric data that matches the digital representation of the biometric. In various such examples, the identity information may include an assignment of the medical proxy to the person.

In a number of examples, the person may be a proxy assignee and the action may include at least one of obtaining a medical product for a proxy designator, accessing a medical record of the proxy designator, communicating with a medical service provider regarding medical care provided to the proxy designator, or authorizing a medical procedure for the proxy designator. In various examples, the at least one processing unit may verify that the medical proxy authorizes the person to request the action prior to allowing the action to be performed.

In some examples, the at least one processing unit may communicate with a first data store to determine the identity and a second data store to determine that the medical proxy is associated with the identity. In various such examples, the first data store may be an identity system data store and the second data store may be a medical records data store.

In some implementations, a biometric medical proxy system may include at least one non-transitory storage medium that stores instructions and at least one processing unit. The at least one processing unit may execute the instructions to receive a request to perform an action that requires an authorization from a particular person, obtain a digital representation of a biometric for the authorization, determine that an identity associated with the digital representation of the biometric is for a different person, and, upon determining that the identity is associated with a medical proxy regarding the action for the particular person, allow the action to be performed.

In various examples, the action may be picking up a prescription medical product. In some such examples, the at least one processing unit may prompt the different person to select among prescription medical products available to pick up for multiple people for whom the different person has medical proxies.

In some examples, the at least one processing unit may determine that the identity is associated with the medical proxy by transmitting a communication to the particular person to assign the medical proxy to the different person for the action. In a number of such examples, the digital representation of the biometric may be a digital representation of a first biometric and the at least one processing unit may receive a digital representation of a second biometric from the particular person to assign the medical proxy to the different person for the action. In various examples, the at least one processing unit may perform the action.

In a number of implementations, a biometric medical proxy system may include at least one non-transitory storage medium that stores instructions and at least one processing unit. The at least one processing unit may execute the instructions to obtain a digital representation of a first biometric of a proxy designator and a request to designate a proxy assignee as a medical proxy for the proxy designator, determine a first identity of the proxy designator using the digital representation of the first biometric, obtain a digital representation of a second biometric from the proxy assignee, and assign the medical proxy to a second identity of the proxy assignee associated with the digital representation of the second biometric.

In some examples, the request to designate the proxy assignee as the medical proxy for the proxy designator may be the digital representation of the second biometric obtained by the at least one processing unit after the at least one processing unit obtains the digital representation of the first biometric. In various examples, the request to designate the proxy assignee as the medical proxy for the proxy designator may specify contact information for the proxy assignee and the at least one processing unit may use the contact information to request the digital representation of the second biometric from the proxy assignee. In some such examples, the contact information may specify an electronic device from which the digital representation of the second biometric can be obtained and that is associated with the proxy assignee. In other such examples, the contact information may be used to determine the second identity, the second identity may include a specification of an electronic device from which the digital representation of the second biometric can be obtained and that is associated with the proxy assignee, and the at least one processing unit may use the specification to request the digital representation of the second biometric from the proxy assignee.

In some examples, the request to designate the proxy assignee as the medical proxy for the proxy designator may specify an action to which the medical proxy applies. In various examples, the at least one processing unit may receive a request to perform an action that requires an authorization, receive the authorization from the proxy assignee, and, upon determining that the authorization includes the digital representation of the second biometric, allow the action to be performed.

Although the above illustrates and describes a number of embodiments, it is understood that these are examples. In various implementations, various techniques of individual embodiments may be combined without departing from the scope of the present disclosure.

The present disclosure recognizes that biometric and/or other personal data is owned by the person from whom such biometric and/or other personal data is derived. This data can be used to the benefit of those people. For example, biometric data may be used to conveniently and reliably identify and/or authenticate the identity of people, access securely stored financial and/or other information associated with the biometric data, and so on. This may allow people to avoid repeatedly providing physical identification and/or other information.

The present disclosure further recognizes that the entities who collect, analyze, store, and/or otherwise use such biometric and/or other personal data should comply with well-established privacy policies and/or privacy practices. Particularly, such entities should implement and consistently use privacy policies and practices that are generally recognized as meeting or exceeding industry or governmental requirements for maintaining security and privately maintaining biometric and/or other personal data, including the use of encryption and security methods that meets or exceeds industry or government standards. For example, biometric and/or other personal data should be collected for legitimate and reasonable uses and not shared or sold outside of those legitimate uses. Further, such collection should occur only after receiving the informed consent. Additionally, such entities should take any needed steps for safeguarding and securing access to such biometric and/or other personal data and ensuring that others with access to the biometric and/or other personal data adhere to the same privacy policies and practices. Further, such entities should certify their adherence to widely accepted privacy policies and practices by subjecting themselves to appropriate third party evaluation.

Additionally, the present disclosure recognizes that people may block the use of, storage of, and/or access to biometric and/or other personal data. Entities who typically collect, analyze, store, and/or otherwise use such biometric and/or other personal data should implement and consistently prevent any collection, analysis, storage, and/or other use of any biometric and/or other personal data blocked by the person from whom such biometric and/or other personal data is derived.

As described above and illustrated in the accompanying figures, the present disclosure relates to setting and verifying medical proxies using biometric identification systems. A proxy assignee may be granted a medical proxy for a proxy designator by the proxy designator providing a digital representation of a biometric for the proxy designator to a biometric identification system and/or specifying the rights to be granted and the identity of the proxy assignee to whom to grant the rights. The proxy assignee may accept the medical proxy by providing a digital representation of a biometric for the proxy assignee, and the medical proxy may then be associated with identity information stored for the proxy assignee. When the proxy assignee requests to perform an action for the proxy designator that requires an authorization, the proxy assignee may be identified using the digital representation of the biometric for the proxy assignee. Upon determining that the medical proxy is associated with the identity (and/or verifying that the medical proxy authorizes the proxy assignee for the action for the proxy designator), the action may be performed.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A biometric medical proxy system, comprising:
at least one non-transitory storage medium that stores instructions; and
at least one processing unit that executes the instructions to:
receive a request to perform an action that requires an authorization;
determine that the authorization is obtained from a person asserting to have a medical proxy;
determine an identity of the person using a digital representation of a biometric; and
upon determining that the medical proxy is associated with the identity, allow the action to be performed.

2. The biometric medical proxy system of claim 1, wherein the at least one processing unit determines the identity of the person by retrieving identity information that is associated with biometric data that matches the digital representation of the biometric.

3. The biometric medical proxy system of claim 2, wherein the identity information includes an assignment of the medical proxy to the person.

4. The biometric medical proxy system of claim 1, wherein the person is a proxy assignee and the action comprises at least one of:
obtaining a medical product for a proxy designator;
accessing a medical record of the proxy designator;
communicating with a medical service provider regarding medical care provided to the proxy designator; or
authorizing a medical procedure for the proxy designator.

5. The biometric medical proxy system of claim 1, wherein the at least one processing unit verifies that the medical proxy authorizes the person to request the action prior to allowing the action to be performed.

6. The biometric medical proxy system of claim 1, wherein the at least one processing unit communicates with a first data store to determine the identity and a second data store to determine that the medical proxy is associated with the identity.

7. The biometric medical proxy system of claim 6, wherein the first data store is an identity system data store and the second data store is a medical records data store.

8. A biometric medical proxy system, comprising:
at least one non-transitory storage medium that stores instructions; and
at least one processing unit that executes the instructions to:
receive a request to perform an action that requires an authorization from a particular person;
obtain a digital representation of a biometric for the authorization;
determine that an identity associated with the digital representation of the biometric is for a different person; and
upon determining that the identity is associated with a medical proxy regarding the action for the particular person, allow the action to be performed.

9. The biometric medical proxy system of claim 8, wherein the action comprises picking up a prescription medical product.

10. The biometric medical proxy system of claim 9, wherein the at least one processing unit prompts the different person to select among prescription medical products available to pick up for multiple people for whom the different person has medical proxies.

11. The biometric medical proxy system of claim 8, wherein the at least one processing unit determines that the identity is associated with the medical proxy by transmitting a communication to the particular person to assign the medical proxy to the different person for the action.

12. The biometric medical proxy system of claim 11, wherein:
the digital representation of the biometric is a digital representation of a first biometric; and
the at least one processing unit receives a digital representation of a second biometric from the particular person to assign the medical proxy to the different person for the action.

13. The biometric medical proxy system of claim 8, wherein the at least one processing unit performs the action.

14. A biometric medical proxy system, comprising:
at least one non-transitory storage medium that stores instructions; and
at least one processing unit that executes the instructions to:
obtain a digital representation of a first biometric of a proxy designator and a request to designate a proxy assignee as a medical proxy for the proxy designator;
determine a first identity of the proxy designator using the digital representation of the first biometric;
obtain a digital representation of a second biometric from the proxy assignee; and
assign the medical proxy to a second identity of the proxy assignee associated with the digital representation of the second biometric.

15. The biometric medical proxy system of claim 14, wherein the request to designate the proxy assignee as the medical proxy for the proxy designator comprises the digital representation of the second biometric obtained by the at least one processing unit after the at least one processing unit obtains the digital representation of the first biometric.

16. The biometric medical proxy system of claim 14, wherein:
the request to designate the proxy assignee as the medical proxy for the proxy designator specifies contact information for the proxy assignee; and
the at least one processing unit uses the contact information to request the digital representation of the second biometric from the proxy assignee.

17. The biometric medical proxy system of claim 16, wherein the contact information specifies an electronic device from which the digital representation of the second biometric can be obtained and that is associated with the proxy assignee.

18. The biometric medical proxy system of claim 16, wherein:
the contact information is used to determine the second identity;
the second identity includes a specification of an electronic device from which the digital representation of the second biometric can be obtained and that is associated with the proxy assignee; and
the at least one processing unit uses the specification to request the digital representation of the second biometric from the proxy assignee.

19. The biometric medical proxy system of claim 14, wherein the request to designate the proxy assignee as the medical proxy for the proxy designator specifies an action to which the medical proxy applies.

20. The biometric medical proxy system of claim 14, wherein the at least one processing unit:
receives a request to perform an action that requires an authorization;
receives the authorization from the proxy assignee; and
upon determining that the authorization includes the digital representation of the second biometric, allows the action to be performed.
